(19) United States
(12) Patent Application Publication
(10) Pub. No.: US 2022/0409442 A1
(43) Pub. Date: Dec. 29, 2022

(54) ABSORBENT SANITARY ARTICLE AND A METHOD FOR PRODUCING THE SAME

(71) Applicant: Fameccanica.Data S.p.A., San Giovanni Teatino (Chieti) (IT)

(72) Inventors: Diego GUALTIERI, San Giovanni Teatino (Chieti) (IT); Alessandro CIPRIANI, San Giovanni Teatino (Chieti) (IT)

(21) Appl. No.: 17/842,853

(22) Filed: Jun. 17, 2022

(30) Foreign Application Priority Data
Jun. 24, 2021 (EP) 21181365.4

(51) Int. Cl. A61F 13/49; A61F 13/494

(52) U.S. Cl. CPC A61F 13/49001; A61F 13/49453; A61F 2013/49076

(57) ABSTRACT

An absorbent sanitary article includes a chassis having a front section and a rear section, a micro-hook front panel integrally formed on a portion of a backsheet in the front section of the chassis and two back side panels attached to the rear section of the chassis and having respective micro-loop areas configured for forming a hook-and-loop connection with the micro-hook front panel.

### FIELD OF THE INVENTION

The present invention relates to absorbent sanitary articles, such as, for example, diapers, training pants, absorbent sanitary products for incontinent adults, etc.

The present invention also relates to a method for producing absorbent sanitary articles.

### PRIOR ART

An absorbent sanitary article typically has a structure that comprises a rectangular-shaped central body or chassis having a front section and a rear section. The chassis is normally formed by a permeable topsheet intended to come into contact with the user's skin when the article is worn, an impermeable backsheet and an absorbent core sandwiched between the topsheet and the backsheet. The front and rear section of the chassis are normally closed around the user's waist by means of hook-and-loop fasteners, better known as Velcro® fastening devices.

In many cases the absorbent sanitary article has a front panel of micro-loop material attached to the front section of the chassis and one pair of elastic back side panels which extend laterally from opposite sides of the rear section of the chassis. The elastic back side panels are provided with fastening tabs, typically comprising micro-hooks pads, which can be releasably attached to the front panel of micro-loop material for closing the absorbent sanitary article around the waist of the user.

The front panel of a hook-and-loop fastener is formed of a fibrous material, for example polypropylene, provided on an upper surface with micro-loops suitable for engaging with the micro-hooks of the side panels. The loop-material is normally fixed to a support typically consisting of a film of plastic material or of a non-woven polypropylene or the like.

EP-A-2540272 describes a multi-layer material for diaper front panels, comprising a support formed of a polyolefinic film and a layer of loop-material formed by a non-woven polyolefinic material. The support and the layer of loop-material are normally secured together by glue.

In conventional solutions, loop material attached to the support is supplied in reels, which are cut transversely to form the front panels which are applied to the outer surface of respective backsheet in the respective front sections. The front panels are normally connected to the backsheet by glue or by ultrasonic welding. Welding is generally not suitable because it can compromise the impermeability of the backsheet.

The backsheet of a diaper is normally a multi-layer composite structure including an inner impermeable film and an outer layer made of a non-woven web connected to the impermeable film by a layer of glue. The front panel is applied on the outer surface of the non-woven web of the backsheet, usually by a layer of glue.

The production of prior art elastic back side panels requires micro-hook pads produced starting from continuous tapes, usually made of relatively rigid thermoplastic material, having continuous or intermittent micro-hook formations integrally formed thereon. The continuous tape with integrally formed micro-hook formations is cut to form a plurality of discrete micro-hook pads. Such discrete micro-hook pads are spaced by a desired pitch and are fixed in spaced positions on a surface of a continuous non-woven support web. The continuous non-woven support web with the micro-hook pads fixed thereon is then cut to form fastening tabs each including a non-woven support web of soft material and a micro-hook pad of relatively rigid thermoplastic material fixed on a surface of the non-woven support web by glue or by welding.

The continuous tapes of micro-hook and micro-loop material are manufactured by suppliers specialized in manufacturing hook-and-loop fasteners. The micro-hook ad micro-loop tapes are packaged in reels which are delivered to the manufacturing plants of absorbent sanitary articles. In the machines for manufacturing absorbent sanitary articles the reels of continuous tapes with integrally formed micro-hook and micro loop formations are unwound, cut, spaced, and fixed to the continuous non-woven support web. All these operations require complex and expensive dedicated equipment which increase the complexity of the machines for manufacturing absorbent sanitary articles. Since the cost of the micro-hook tapes is high, the dimension of the micro-hook pads is kept to the minimum possible, which requires complex equipment for cutting the continuous micro-hook tapes in very small pieces and for feeding such small pieces with high precision to the continuous non-woven support web.

A further drawback of the prior art is that the micro-hook fastening pads of relatively rigid plastic material are positioned on the inner surfaces of the back side panels, which face the skin of the user when the diaper is applied to the user. This may give rise to an unpleasant stiffness and sharpness perception during the application of the sanitary article to the wearer. Also, the user may get scratches or abrasions due to the contact with sharp and rigid micro-hook pads.

WO2010/085492 discloses an apparatus and process for forming micro-hooks on a substrate for use as hook-type fasteners in touch fastening systems, wherein vibration energy is used to soften a substrate which is positioned between a mold and a source of vibration. The mold includes a plurality of cavities into which the softened substrate is forced to form the projections. The substrate may comprise a film, sheet, web, composite, laminate, etc. The source of vibration may be an ultrasonic horn. The process to form such micro-hooks may be operated in a continuous, semi-continuous or intermittent manner.

### OBJECT AND SUMMARY OF THE INVENTION

The object of the present invention is to provide an absorbent sanitary article which overcomes the problems of the prior art.

According to the invention, this object is achieved by an absorbent sanitary article having the features of claim 1.

According to another aspect, the invention relates to a method for manufacturing absorbent sanitary articles having the features of claim 7.

The claims form an integral part of the technical disclosure provided here in relation to the invention.

### BRIEF DESCRIPTION OF THE DRAWINGS

The present invention will now be described with reference to the attached drawings, provided purely by way of non-limiting example, wherein:

FIG. 1 is a perspective view of an absorbent sanitary article according to the present invention,

FIGS. 2-5 are schematic plan views showing different embodiments of absorbent sanitary articles according to the present invention,

FIGS. 6 and 7 are schematic perspective views showing a method for manufacturing the backsheet of the absorbent sanitary articles according to the present invention,

FIG. 8 is a schematic plan view taken along the arrow VIII of FIG. 6,

FIG. 9 is a schematic cross-section taken along the line IX-IX of FIG. 8, and

FIG. 10 is a schematic cross-section taken along the line X-X of FIG. 2.

It should be appreciated that the attached drawings are schematic and not to scale with respect to real products. Various figures may not be represented in the same scale. Also, in various figures some elements may not be shown to better show other elements.

### DETAILED DESCRIPTION

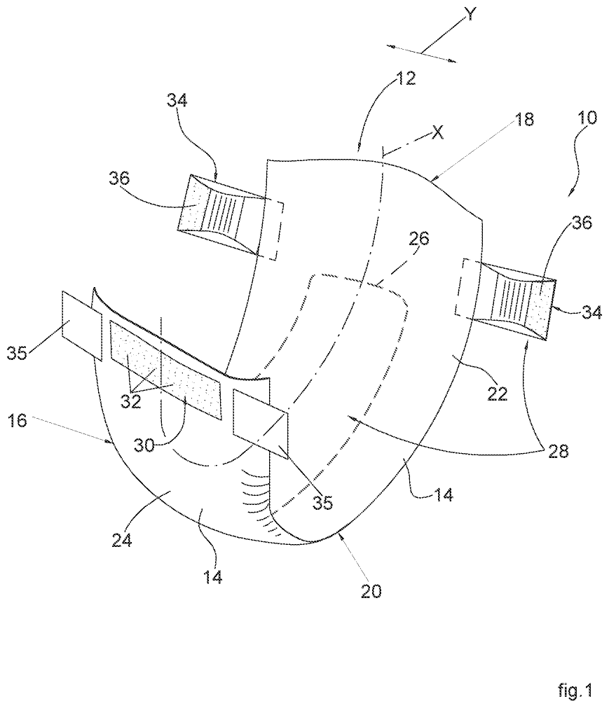

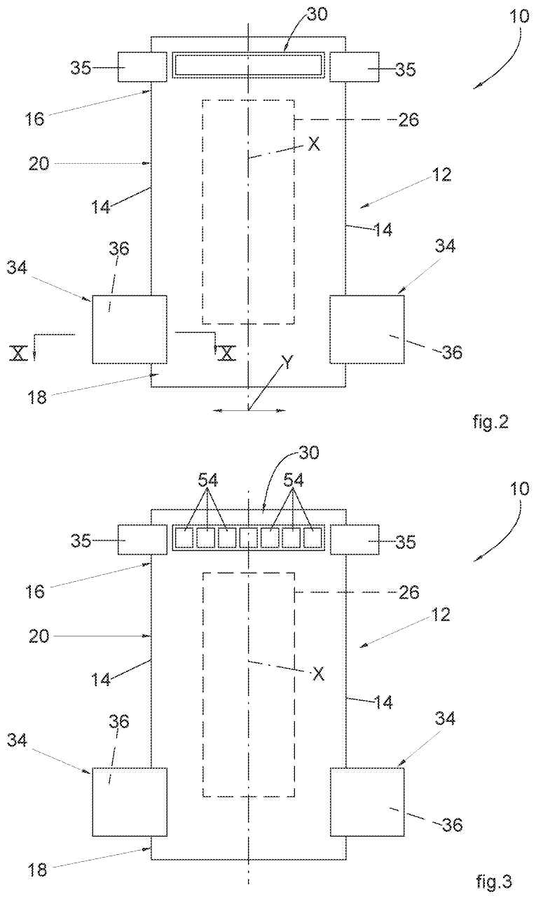

With reference to FIGS. 1-5, numeral 10 indicates an absorbent sanitary article according to the present invention. FIG. 1 shows the absorbent sanitary article 10 in the configuration in which it is worn. FIGS. 2-5 show various embodiments of absorbent sanitary articles 10 according to the present invention in a flat configuration.

The absorbent sanitary article 10 comprises a chassis 12 elongated along a longitudinal axis X. The chassis 12 has two side edges 14 parallel to the longitudinal axis X, a front section 16 and a rear section 18. The front section 16 and the rear section 18 in use are closed around the user's waist. Between the front section 16 and the rear section 18 a groin section 20 extends which, in use, is arranged between the legs of the user.

The chassis 12 comprises a topsheet 22 made of a permeable material which, in use, is in contact with the user's skin, an impermeable backsheet 24 and an absorbent core 26 sandwiched between the topsheet 22 and the backsheet 24. The chassis 12 may comprise additional components as usual in the field of absorbent sanitary articles, such as elastic leg cuffs, acquisition and diffusion layers, etc.

The absorbent sanitary article 10 comprises a hook-and-loop fastening device 28 configured for fastening the front section 16 and the rear section 18 to each other.

The hook-and-loop fastening device 28 comprise a micro-hook front panel 30 located in the front section 16 of the chassis 12. The micro-hook front panel 30 includes a plurality of micro-hooks 32 integrally formed on a portion of the outer surface of the backsheet 24, which are formed as it will be disclosed in the following.

The hook-and-loop fastening device 28 comprise two back side panels 34 attached to the rear section 18 of the chassis 12 and extending laterally beyond respective side edges 14 in a transverse direction Y orthogonal to the longitudinal axis X. The side panels 34 have respective micro-loop areas 36 configured for forming a releasable surface connection with the micro-hook front panel 30. The micro-loop areas 36 may be formed by a portion of a non-woven web. The side panels 34 may be elastically stretchable the transverse direction Y.

The absorbent sanitary article 10 may comprises a pair of front side panels 35 projecting laterally from opposite sides of the front section 16, having the function of facilitating attachment of the back side panels 34 to the micro-hook front panel 30 when the absorbent sanitary article 10 is closed around the user's waist.

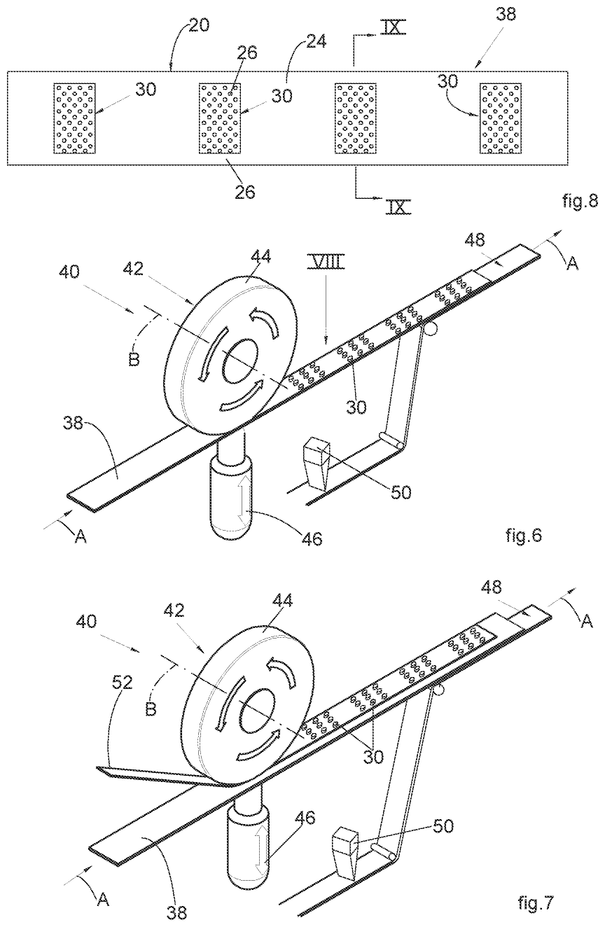

The backsheet 24 with the integral micro-hook front panel 30 may be produced by a method disclosed in WO2010/085492 and schematically shown in FIGS. 6 and 7.

With reference to FIG. 6, a continuous non-woven web 38 moving in its longitudinal direction A passes through a micro-hook forming unit 40. The micro-hook forming unit 40 comprises a moulding roller 42 rotating about an axis B transversal to the longitudinal direction A of the continuous non-woven web 38. The moulding roller 42 has a plurality of micro-cavities open on its outer cylindrical surface 44. The micro-hook forming unit 40 comprises an ultrasonic horn 46 which compresses the continuous non-woven web 38 against the outer cylindrical surface 44 of the moulding roller 42 as the continuous non-woven web 38 moves in the direction A.

The vibrating energy produced by the ultrasonic horn 46 liquefies or fluidifies locally the non-woven material of the continuous non-woven web 38, which penetrates in a fluid or liquid state in the micro-cavities of the moulding roller 42. The material of the non-woven web 38 which penetrates in the cavities of the molding roller 29 cools in contact with the walls of the micro-cavities and forms micro-hook front panels 30 integral with the continuous non-woven web 38.

As shown in FIG. 8, at the exit of the micro-hook forming unit 40 the continuous non-woven web 38 has an array of micro-hook front panels 30 spaced apart from each other in the longitudinal direction A.

The array of spaced apart micro-hook front panels 30 may be obtained by providing on the outer surface 44 of the moulding roller 42 an intermittent pattern of micro-cavities. Alternatively, the moulding roller 42 may have a continuous pattern of micro-cavities and the array of spaced apart micro-hook front panels 30 may be formed by intermittently turning off the ultrasonic horn 46. As a further alternative, the moulding roller 42 may have a continuous pattern of micro-cavities and the array of spaced apart micro-hook front panels 30 may be formed by intermittently detaching the ultrasonic horn 46 from the outer cylindrical surface 44 of the moulding roller 42.

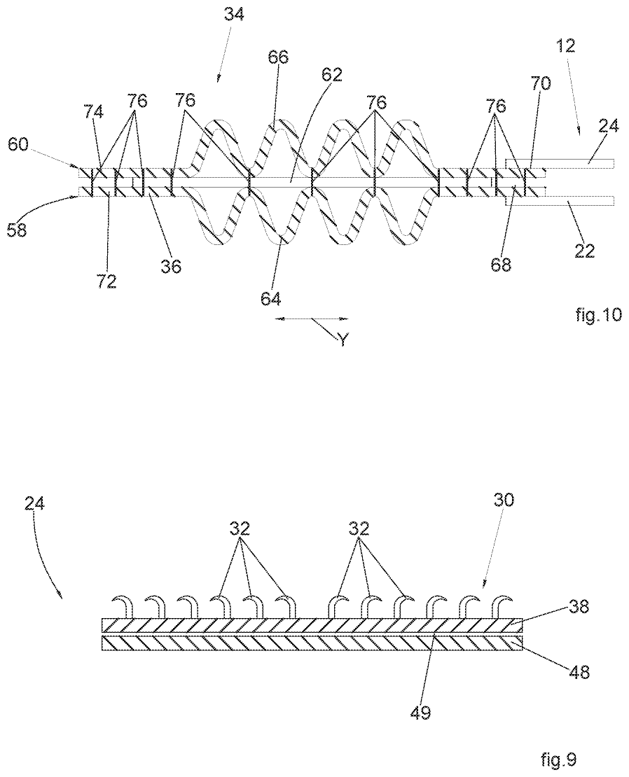

At the exit of the micro-hook forming unit 40 the continuous non-woven web 38 with the array of spaced apart integrally formed micro-hook front panels 30 is overlapped and fixed to a continuous impermeable film 48 moving in the same direction A as the continuous non-woven web 38. With reference to FIGS. 6, 7 and 9, the continuous non-woven web 38 and the continuous impermeable film 48 may be fixed to each other by a layer of glue 49 which may be applied on a surface of the continuous impermeable film 48 by a glue dispenser 50.

The continuous non-woven web 38 and the continuous impermeable film 48 form a continuous backsheet comprising an inner impermeable film 48 and an outer non-woven layer 38 fixed to the inner impermeable film 48 e.g. by a layer of glue 49, wherein an array of micro-hook front panels 30 is integrally formed on an outer surface of the outer non-woven layer 38.

In order to form micro-hooks with dimensions sufficient for an effective hook-and-loop connection, the continuous non-woven layer 38 should have a weight of 20-120 g/m².

With reference to FIG. 7, in a possible embodiment, an additional continuous web 52 may be applied on the continuous non-woven web 38 before passing the two webs 38, 52 through the micro-hook forming unit 42. The additional continuous web 52 may be a thermoplastic film or a non-woven web of a material different or identical to the material of the continuous non-woven web 38. After passing through the micro-hook forming unit 40, the additional continuous web 52 and the continuous non-woven web 38 form a unitary layer with integrally formed micro-hook front panels 30. The use of an additional web 52 may allow a reduction of the weight of the continuous non-woven web 38. For instance, in a possible embodiment two continuous non-woven webs 38, 52 may be used, each having a weight of 40 g/m2, which is a standard weight of non-woven webs in the field of absorbent sanitary articles.

In a possible embodiment, the micro-hook forming unit 40 may be a thermomechanical forming unit, in which the material of the non-woven web 38, or webs 38, 52, is locally liquified or fluidified by heating and compression.

With reference to FIG. 3, in a possible embodiment the micro-hooks 32 of the micro-hook front panel 30 may be arranged in a plurality of discrete pads 54.

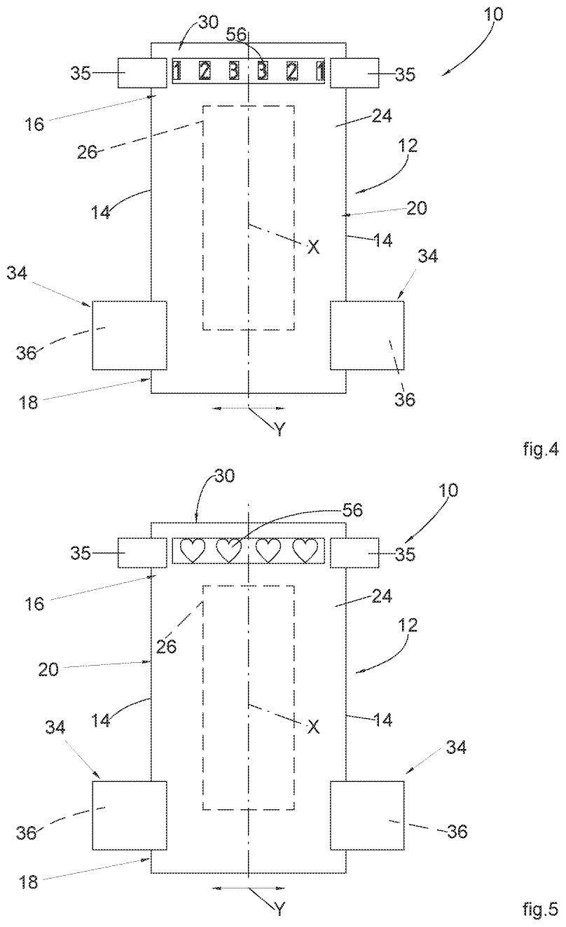

With reference to FIGS. 4 and 5, in a possible embodiment the micro-hooks 32 of the micro-hook front panel 30 may be arranged in a pattern 56 forming images, writings or numbers. The numbers may provide a visual indication of the position for attaching the back side panels 34. Images and writings may indicate the trademark of the producer or the trade-name of the article.

With reference to FIG. 10, the back side panel 34 may comprise a first non-woven web 58, a second non-woven web 60 and an elastic film 62 sandwiched between the first and second non-woven web 58, 60. The elastic film 18 is elastically stretchable in a transversal direction Y.

The first and second non-woven web 58, 60 have respective pleated central portions 64, 66, respective non-pleated proximal portions 68, 70 and respective non-pleated distal portions 72, 74. The first and second non-woven web 58, 60 and the elastic film 62 are fixed to each other, e.g. by a pattern of spot welds 76. The non-pleated proximal portions 68, 70 are fixed to the chassis 12. The surface of the non-pleated distal portion 72 forms a micro-loop area 36 which forms the loop portion of a hook-and-loop fastening connection.

The back side panels 34 have a soft feeling to the touch because they do not have micro-hooks. The back side panels 34 do not require the application of discrete micro-hook pads and this involves considerable advantages in that there is no need for complex and expensive devices for applying discrete elements at the distal portions of the side panels 34.

The micro-hook front panel 30 is relatively soft as compared to the prior art micro-hook pads, because the micro-hooks 32 are obtained starting from non-woven material, which is softer than the thermoplastic material forming the traditional micro-hooks of hook-and-loop fastening devices. The micro-hook front panel 30 is located on an outer surface of the absorbent sanitary article, which does not contact the skin of the user when the absorbent sanitary article is applied to the user.

The hook-and-loop fastening device of the absorbent sanitary article according to the present invention is integrally formed on materials which are already present in the structure of the absorbent sanitary article. The hook-and-loop fastening device 28 of the absorbent sanitary article according to the present invention does not require reels of micro-hook ad micro-loop tapes to be transported and delivered to the manufacturing plants of absorbent sanitary articles. The invention has therefore considerable advantages regarding lower costs of materials, simpler manufacturing machines and an improved sustainability.

The absorbent sanitary articles 10 according to the present invention may be manufactured by a method comprising:
integrally forming an array of micro-hook front panels 30 spaced apart from each other on an outer surface of a continuous backsheet 24,
sandwiching absorbent cores 26 between a continuous topsheet 22 and the continuous backsheet 24 with integrally formed micro-hook front panels 30, so as to form a chain of chassis 12, wherein the micro-hook front panels 30 are positioned in respective front sections 16 of respective chassis 12, and
providing said chain of chassis 12 with an array of pair of back side panels 34 attached to respective rear sections 18 of respective chassis 12 and extending laterally beyond respective side edges 14 in a transverse direction (Y) orthogonal to said longitudinal axis (X), wherein the back side panels 34 have respective micro-loop areas 36 configured for forming a hook-and-loop connection with the micro-hook front panels 30.

The chain of chassis 12 is then cut transversely to form individual absorbent sanitary articles 10.

The formation of the micro-hook front panels 30 on the outer surface of the continuous backsheet 24 may comprise:
providing an inner impermeable film 48 and an outer non-woven layer 38, 52,
integrally forming the micro-hook front panels 30 on portions of the outer surface of the outer non-woven layer 38, 52, and
   fixing the outer non-woven layer 38, 52 with integrally formed micro-hook front panels 30 to the inner impermeable film 48.

Of course, without prejudice to the principle of the invention, the details of construction and the embodiments can be varied, even significantly, with respect to those illustrated here without departing from the scope of the invention as defined by the following claims.

1. An absorbent sanitary article, comprising:
a chassis having a longitudinal axis, two side edges, a front section and a rear section, the chassis including a topsheet, a backsheet having an outer surface, and an absorbent core sandwiched between the topsheet and the backsheet,
a micro-hook front panel including a plurality of micro-hooks integrally formed on a portion of the outer surface of said backsheet in said front section of the chassis, and
two back side panels attached to said rear section of the chassis and extending laterally beyond the respective two side edges in a transverse direction orthogonal to said longitudinal axis, wherein said two back side panels are elastically stretchable in said transverse direction, wherein each of said two back side panels comprises a first and a second non-woven web, having respective pleated central portions and respective non-pleated proximal and distal portions on opposite sides of said pleated central portions, and an elastic film sandwiched between said pleated central portions of said first and a second non-woven web, and wherein an outer surface of the distal portion of said first non-woven web forms a micro-loop area configured for forming a hook-and-loop connection with said micro-hook front panel.

2. The absorbent sanitary article of claim 1, wherein said backsheet comprises an inner impermeable film and an outer non-woven layer fixed to the inner impermeable film, wherein said micro-hook front panel is integrally formed on said outer non-woven layer.

3. The absorbent sanitary article of claim 2, wherein said outer non-woven layer has a weight of 20-120 g/m².

4. The absorbent sanitary article of claim 2, wherein said outer non-woven layer comprises two overlapped non-woven webs.

5. The absorbent sanitary article of claim 1, wherein the plurality of micro-hooks of said micro-hook front panel are arranged in a pattern forming images, and/or writings and/or numbers.

6. The absorbent sanitary article of claim 1, wherein the plurality of micro-hooks of the micro-hook front panel are arranged in a plurality of discrete pads.

7. A method for producing absorbent sanitary articles, comprising:
integrally forming an array of micro-hook front panels spaced apart from each other on an outer surface of a continuous backsheet,
sandwiching absorbent cores between a continuous topsheet and the continuous backsheet with integrally formed micro-hook front panels, so as to form a chain of chassis, wherein the micro-hook front panels are positioned in respective front sections of the chain of chassis.
forming an array of pairs of back side panels elastically stretchable in a transverse direction, wherein each of said back side panels comprises first and second non-woven webs, having respective pleated central portions and respective non-pleated proximal and distal portions on opposite sides of said pleated central portions, and an elastic film sandwiched between said pleated central portions of said first and second non-woven webs, wherein an outer surface of the distal portion of said first non-woven web forms a micro-loop area configured for forming a hook-and-loop connection with the micro-hook front panels,
attaching said proximal portions of said pairs of back side panels to respective rear sections of the chain of chassis such that said distal portions in said transverse direction extend laterally beyond respective side edges of the respective rear sections of the chain of chassis, and
transversely cutting the chain of chassis to form individual absorbent sanitary articles.

8. The method of claim 7, wherein integrally forming said array of micro-hook front panels on the outer surface of the continuous backsheet comprises:
providing an inner impermeable film and an outer non-woven layer,
integrally forming said array of micro-hook front panels on portions of an outer surface of said outer non-woven layer, and
fixing said outer non-woven layer with integrally formed micro-hook front panels to the inner impermeable film.

9. The method of claim 8, wherein said outer non-woven layer has a weight of 20-120 g/m².

10. The method of claim 8, comprising forming said outer non-woven layer by overlapping two non-woven webs.

11. The method of claim 7, comprising arranging micro-hooks of each of said micro-hook front panels in a pattern forming images, and/or writings and/or numbers and/or a plurality of discrete pads.

12. The method of claim 7, wherein micro-hooks of said micro-hook front panels are integrally formed in a non-woven layer by locally liquifying or fluidifying the non-woven layer and inserting the liquified or fluidified non-woven material into cavities of a molding roller.

13. The method of claim 12, wherein locally liquifying or fluidifying the non-woven material is obtained by subjecting a non-woven layer to ultrasonic compression or to thermomechanical compression.
